SEQUENCE LISTING (8 sequences)

SEQ ID NO: 1 - DNA, 36 bp, synthetic construct
  source 1..36: mol_type = other DNA
atccaatttagagtctcacctgcttcaactaatgaa

SEQ ID NO: 2 - DNA, 36 bp, synthetic construct
  source 1..36: mol_type = other DNA
gactctaaattggattacagcggtggcagctgcagc

SEQ ID NO: 3 - DNA, 39 bp, synthetic construct
  source 1..39: mol_type = other DNA
gtacgcaattcacgctcatcacctgcttcaactaatgaa

SEQ ID NO: 4 - DNA, 39 bp, synthetic construct
  source 1..39: mol_type = other DNA
tgagcgtgaattgcgtactacagcggtggcagctgcagc

SEQ ID NO: 5 - protein, 390 aa, Homo sapiens
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 6 - protein, 390 aa, synthetic construct
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVIQFRVSPTSTNEEFHCNHPFLFFIRQNKTNSILFYGRFSSP

SEQ ID NO: 7 - protein, 22 aa, Homo sapiens
EEGAEAAAATAVVGFGSSPTST

SEQ ID NO: 8 - protein, 22 aa, synthetic construct
EEGAEAAAATAVIQFRVSPTST

(19) United States
(12) Patent Application Publication
(10) Pub. No.: US 2024/0350597 A1
(43) Pub. Date: Oct. 24, 2024

(54) PROTEASE INHIBITORS

(71) Applicant: University College Dublin, Dublin (IE)

(72) Inventors: D. Margaret Worrall, Belfield (IE); Saravjeet Singh, Belfield (IE)

(21) Appl. No.: 18/138,612

(22) Filed: Apr. 24, 2023

(51) Int. Cl. A61K 38/55; A61P 31/14; C07K 14/81

(52) U.S. Cl. CPC A61K 38/55; A61P 31/14; C07K 14/8121

(57) ABSTRACT

The invention relates to a serine protease inhibitor (Serpin) comprising a modified Reactive Centre Loop (RCL), wherein the modified RCL comprises a transmembrane serine protease 2 (TMPRSS2) inhibitory sequence having one or more amino acid substitutions at positions P4 to P1′. The invention also relates to a method of treating and/or preventing a condition in a subject in need thereof, where the TMPRSS2 activity is implicated in said condition, the method comprising administering the Serpin of the present invention.

### FIELD OF THE INVENTION

The present invention relates to a serine protease inhibitor (Serpin) that specifically targets TMPRSS2. The invention also relates to a method of treating conditions where TMPRSS2 activity is implicated by administering a composition comprising a Serpin that specifically targets TMPRSS2.

### SEQUENCE LISTING

The instant application contains a Sequence Listing which has previously been submitted electronically in xml format on Jun. 6, 2023, and is hereby incorporated by reference in its entirety. Said previously-submitted xml copy is named 119452-000015_Sequence_Listing.xml, created on and is 4 KB in size.

### BACKGROUND OF THE INVENTION

Serine proteases are essential proteins that drive many physiological processes, including tissue remodelling, vascular haemostasis and inflammation. However, excessive activity can cause disease and therefore the activity of serine proteases must be tightly regulated.

Serine protease inhibitors (Serpins) are the major human class of protease inhibitors. They have a highly conserved secondary structure comprising three β-sheets (named A-C) and nine α-helices (named A-I). Although serpins typically target serine proteases, some inhibit other protease classes, often cysteine proteases, and the specificity for their targets is dictated by the exposed Reactive Centre Loop (RCL), a flexible loop protruding from the main body of the protein.

The RCL acts as a pseudosubstrate that facilitates the inhibition of serine proteases by serpins. The serine protease catalyses the cleavage of the RCL via nucleophilic attack, forming an acyl-enzyme intermediate. However, cleavage of the P1-P1′ scissile bond of the RCL causes the serpin to undergo a conformational change, whereby the RCL inserts itself within the strands of β-sheet A. This motion distorts the active site of the serine protease and prevents the hydrolysis of the acyl-enzyme intermediate therefore trapping the protein in this covalently bound state.

Transmembrane Serine Proteas 2 (TMPRSS2) is a type II transmembrane serine protease expressed in epithelial cells e.g., in respiratory and prostate tissues, and has been implicated in the pathogenesis of viral infections as well as prostate cancer cell growth, invasion and metastasis.

Recent evidence implicates TMPRSS2 in the pathogenesis of SARS-CoV-2. The entry of the virus into host cells is dependent on the spike (S) protein, which facilitates binding to the angiotensin converting enzyme (ACE2) receptor and fusion of the lipid envelope with the host cell membranes. The S protein is produced as an inactive precursor that must be cleaved at two separate sites by host cell proteases to facilitate membrane fusion and cell entry. Cleavage at the S1/S2 site initiates the engagement between the spike receptor binding domain and the ACE2 receptor; this process exposes a second cleavage site known as S2′. Subsequent S2′ cleavage initiates membrane fusion. TMPRSS2 is thought to be the major host protease involved in S2′ cleavage.

Although the native function of TMPRSS2 remains unclear, TMPRSS2 gene knockout mice show no abnormalities. Small molecule inhibitors of TMPRSS2 have been developed.

It is an object of the present invention to develop a composition targeting TMPRSS2 that addresses one or more of the above identified problems. It is also the object of the present invention to develop a method of treating conditions where TMPRSS2 activity is implicated by administering a composition that specifically targets TMPRSS2.

### SUMMARY OF THE INVENTION

Given that the specificity for a target protease is determined by the sequence of the exposed RCL, serpins could be engineered to inhibit proteases with no known serpin inhibitor. Alternatively, a more selective serpin could be generated that has multiple physiological targets.

According to the first aspect of the present invention, there is provided a serine protease inhibitor (Serpin) comprising a modified Reactive Centre Loop (RCL), wherein the modified RCL comprises a TMPRSS2 inhibitory sequence having one or more amino acid substitutions at positions P4 to P1′.

In accordance with a related aspect of the invention, there is provided a Serpin, wherein the RCL comprises a TMPRSS2 inhibitory sequence having up to four amino acid substitutions at positions P4 to P1′.

Advantageously, the inventors have identified that glutamine (Q) at P3 and arginine (R) at P1 are important residues for TMPRSS2 specificity. Therefore, the TMPRSS2 inhibitory sequence may comprise an amino acid substitution at P3. Preferably, the TMPRSS2 inhibitory sequence comprises glutamine at P3. The TMPRSS2 inhibitory sequence may additionally, or alternatively, comprise an amino acid substitution at P1. Preferably, the TMPRSS2 inhibitory sequence comprises arginine at P1.

The TMPRSS2 inhibitory sequence may comprise an amino acid substitution at P2. Preferably, the TMPRSS2 inhibitory sequence comprises phenylalanine or alanine at P2. Most preferably, the TMPRSS2 inhibitory sequence comprises phenylalanine at P2.

The inventors also identified that mutating P4 and P1′ of the RCL to any hydrophobic residue is also assists with TMPRSS2 specificity. The TMPRSS2 inhibitory sequence may comprise an amino acid substitution at P4 and P1′. Preferably, the TMPRSS2 inhibitory sequence comprises any hydrophobic residue at P4 and P1′.

The RCL sequence is essential for conferring the specificity of the Serpin for its target protease. In a preferred embodiment, the RCL sequence has an amino acid sequence of SEQ ID No: 8. In other embodiments, the RCL has sequence having about 82% identity to SEQ ID No:7. In some embodiments, the RCL has an amino sequence of about 86%, of about 91%, of about 95%, of about 96%, of about 97%, of about 98%, of about 99%, identity of SEQ ID No:8.

Sequence identity may be determined using the on-line algorithm “BLAST” program, which is currently publicly available at http://www.ncbi.nlm.nih.gov/BLAST/. Sequences which have a high degree of identity may be mutants, variants or genetically modified sequences. Deliberate alteration of amino acid sequences may be accomplished by conventional (in vitro) genetic manipulation technologies, such as gene disruption, conjugative transfer, etc. Genetic modification includes introduction of exogenous and/or endogenous DNA sequences. Natural or induced mutations may include at least single base alterations such as deletion, insertion, transversion or other DNA modifications which may result in alteration of the amino acid sequence encoded by the DNA sequence.

The sequence identities are based on the sequence of the Wild Type SerpinB3 (then called SCCA-1) RCL is taken from Schick et al, (1998) (13). For the assessment of identity, the RCL region is generally considered to comprise 20-24 amino acids, so the region used for identity comparison comprises 22 amino acids. The sequence identities are as follows: B3-TMP RCL sequence v. Wild Type RCL=81.8%; B3-TMP RCL sequence v. Wild Type RCL sequence with only 1 amino acid substitution=95.4%; B3-TMP RCL sequence v. Wild Type RCL sequence with 2 amino acid substitutions=90.9%; and B3-TMP RCL sequence v. Wild Type RCL sequence with 3 amino acid substitutions=86.4%.

Any serine protease inhibitor belonging to the Serpin superfamily of proteins can be used as a scaffold upon which the RCL is modified to selectively target TMPRSS2. In some embodiments, the serine protease inhibitor is SerpinB3. In an alternative embodiment, the serine protease inhibitor is alpha-1-antitrypsin (A1AT).

The serine protease inhibitor may have an amino acid sequence of SEQ ID No: 6. The serine protease inhibitor may have a sequence of at least over about 98% sequence identity to SEQ ID No:6. The serine protease inhibitor may have an amino acid sequence of at least over about 98% or at least over about 99% of SEQ ID No:6.

The sequence identities are as follows: B3-TMP sequence v. Wild Type Serpin B3 sequence=98.9%; B3-TMP sequence v Wild Type Serpin B3 sequence with only 1 amino acid substitution=99.7%; B3-TMP sequence v Wild Type Serpin B3 sequence with 2 amino acid substitution=99.48%; and B3-TMP sequence v Wild Type Serpin B3 sequence with 3 amino acid substitution=99.23%.

According to a second aspect of the invention, there is provided an isolated nucleic acid comprising the nucleic acid sequence encoding the Serpin as herein above described.

Advantageously, the inventors have found that the Serpin of the present invention selectively and irreversibly inhibits TMPRSS2. Incubation of the modified Serpin and TMPRSS2 resulted in a 92% inhibition of TMPRSS2 with low off-target binding to other cellular proteases, such as elastase, mammalian trypsin and chymotrypsin. Therefore, the serine protease inhibitor of the present invention will have a wide application wherever inhibition of TMPRSS2 activity is required.

According to a third aspect of the invention, there is provided a use of a composition comprising the Serpin as herein above described to inhibit and/or reduce TMPRSS2 activity in a cell.

Preferably, the cell is a mammalian cell. Most preferably, the cell is a human or animal cell.

According to a fourth aspect of the invention, there is provided a pharmaceutical composition comprising the Serpin as herein above described. A “pharmaceutical composition” is intended to encompass a composition suitable for administration to a subject, such as a mammal, preferably a human.

The pharmaceutical composition may include any pharmaceutically acceptable ingredients well known to those skilled in the art, including, but not limited to, pharmaceutically acceptable carriers, adjuvants, excipients, diluents, fillers, buffers, preservatives, antioxidants, lubricants, stabilisers, solubilisers, surfactants, masking agents and colouring agents.

In general, the routes of administration contemplated by the invention are intended to be, but are not necessarily limited, by intravenous or inhalational routes.

Routes of administration other than inhalation and intravenous administration include, but are not necessarily limited to, intramuscular, intra-tracheal, intrathecal, intracranial, subcutaneous, intradermal, topical, intraperitoneal, intra-arterial (for example, via the carotid artery), spinal or brain delivery, rectal, oral, and other enteral and parenteral routes of administration.

The pharmaceutical composition may be in the form of a liquid or a solid, for example a powder, gel, or paste. An injectable liquid is preferable for intravenous administration. A liquid is preferable for inhalation and may administered in the form of a nasal spray, nasal drops or aerosol administration by nebuliser.

According to a fifth aspect of the invention, there is provided a method of inhibiting TMPRSS2 activity in a cell, the method comprising administering the Serpin as herein above described to a cell.

Preferably, the cell is a mammalian cell. Most preferably, the cell is a human or animal cell.

According to a further aspect of the present invention, there is provided a method of treating and/or preventing a condition in a subject in need thereof, where TMPRSS2 activity is implicated in said condition, the method comprising administering to the subject an effective amount of a serine protease inhibitor (Serpin) comprising a modified reactive centre loop (RCL), wherein the modified RCL comprises a TMPRSS2 inhibitory sequence having one or more amino acid substitutions at positions P4 to P1′.

In accordance with a related aspect of the invention, there is provided a Serpin, wherein the TMPRSS2 inhibitory sequence has up to four amino acid substitutions at positions P4 to P1′.

The TMPRSS2 inhibitory sequence may comprise an amino acid substitution at P3. Preferably, the TMPRSS2 inhibitory sequence comprises glutamine at P3.

The TMPRSS2 inhibitory sequence may comprise an amino acid substitution at P1. Preferably, the TMPRSS2 inhibitory sequence comprises arginine at P1.

The TMPRSS2 inhibitory sequence may comprise an amino acid substitution at P2. Preferably, the TMPRSS2 inhibitory sequence comprises phenylalanine or alanine at P2. Most preferably, the TMPRSS2 inhibitory sequence comprises phenylalanine at P2.

The TMPRSS2 inhibitory sequence may comprise an amino acid substitution at P4 and P1′. Preferably, the TMPRSS2 inhibitory sequence comprises any hydrophobic residue at P4 and P1′.

In a preferred embodiment, the RCL sequence has an amino acid sequence of SEQ ID No: 8. In other embodiments, the RCL has sequence having about 82% identity to SEQ ID No:7. In some embodiments, the RCL has an amino sequence of about 86%, of about 91%, of about 95%, of about 96%, of about 97%, of about 98%, of about 99%, identity of SEQ ID No:8.

The serine protease inhibitor template sequence for RCL mutagenesis can be any protein belonging to the Serpin superfamily. In some embodiments, the serine protease inhibitor is SerpinB3.

In alternative embodiments, the serine protease inhibitor is alpha-1-antitrypsin (A1AT). In some embodiments, the serine protease inhibitor is SerpinB3. In other embodiments, the serine protease inhibitor is alpha-1-antitrypsin (A1AT).

In a preferred embodiment, the serine protease inhibitor has an amino acid sequence of SEQ ID No: 6. In some embodiments, the serine protease inhibitor has a sequence of at least over about 98% sequence identity to SEQ ID No:6. In other embodiments, the serine protease inhibitor has an amino acid sequence of at least over about 98% or at least over about 99% of SEQ ID No:6.

The term “subject” used herein includes any human or nonhuman animal. The term “nonhuman animal” includes all mammals, such as nonhuman primates, sheep, dogs, cats, cows, horses. Preferably, the subject is a human.

A “therapeutically effective amount” refers to the amount of inhibitor that, when administered to a subject for treating a disease, is sufficient to affect such treatment for the disease. The “therapeutically effective amount” will vary depending on the disease and its severity and the age, weight, etc., of the subject to be treated.

The condition may be any condition in which TMPRSS2 activity is implicated and which would benefit from inhibiting or reducing TMPRSS2 activity. The condition may be a pathological or physiological. The condition may be a disease. In certain embodiments, the disease is an infection, and in particular a viral infection. TMPRSS2 is proposed to play a role in coronavirus and influenza virus infectivity by cleaving the Spike (S) protein required for severe acute respiratory syndrome coronavirus 2 (SARS-CoV-2) infection and hemagglutinin required for influenza infection. The inventors of the present invention have advantageously found that administration of the serine protease inhibitor of the present invention prevented SARS-CoV-2 spike (S)-pseudoviral particle entry and downregulated SARS-CoV-2 viral replication, achieving up to 50% inhibition at 6.5 m.

Thus, a related aspect of the present invention is a method of treating a viral infection comprising the administration of an effective amount of the Serpin of the present invention. In some embodiments, the viral infection is caused by a virus from the family of influenza viruses, respiratory syncytial viruses, adenoviruses, parainfluenza viruses, rhinoviruses, enteroviruses, human metapneumoviruses and coronaviruses. Preferably, the viral infection is caused by a coronavirus. Most preferably, the viral infection is caused by severe acute respiratory syndrome coronavirus 2 (SARS-CoV-2).

In some embodiments, the Serpin of the present invention may be administered with one or more compounds effective for the prevention, management, amelioration or treatment of the viral infection.

TMPRSS2 activity has also been implicated in the pathology of prostate cancer and has been linked to prostate cancer growth, invasion and metastasis. Therefore, a related aspect of the present invention is a method of treating cancer comprising the administration of an effective amount of the Serpin of the present invention. Preferably, the cancer is prostate cancer.

In some embodiments, the Serpin of the present invention may be administered with one or more other compounds effective for the prevention, management, amelioration, or treatment of cancer.

### DESCRIPTION OF THE SEVERAL VIEWS OF THE DRAWINGS

The invention is further described in more detail by reference to the following, non-limiting Examples.

FIG. 1 are schematic diagrams showing the structure of the coronavirus spike (S) protein and the SerpinB3 variants of the present invention. (A) is a schematic diagram of the coronavirus S protein with the S1/S2 and S2′ cleavage sites. (B) is a schematic diagram showing the amino acid sequences of the wild type SerpinB3 RCL and the mutated variant generated in this study. The protease cleavage site (P1-P1′) is indicated and amino acid substitutions are indicated by boxes. The image was made in PyMol using the PDB file code 1 QLP.

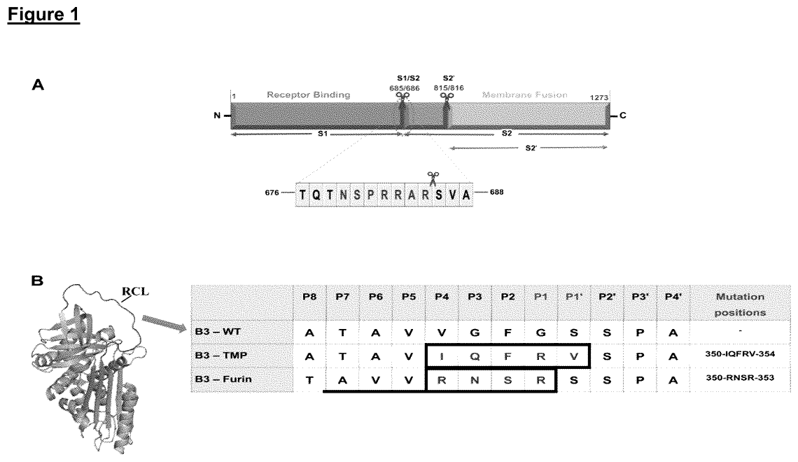

FIG. 2 are graphs showing the change in the target protease activity following incubation with the wild type (WT), B3-TMP and B3-Furin SerpinB3 RCL variants. (A) is a graph showing CatL residual activity with substrate Z-FR-AMC following 2 min incubation with the SerpinB3 variants. (B) is a graph showing TMPRSS2 residual activity with substrate Boc-QAR-AMC following 10 min incubation with the SerpinB3 variants. (C) is a graph showing furin residual activity with substrate Boc-RVRR-AMC following 5 min incubation with the SerpinB3 variants. Residual activity is plotted with uninhibited controls taken as 100% activity. The bar representation results in (A)-(C) are presented as the means±SE performed in triplicate. Statistical analysis was undertaken using a one-way ANOVA with Tukey's comparison test: *** p<0.0005.

FIG. 3 are images of Western blot gels showing the SerpinB3 variants mediated inhibition of recombinant S protein cleavage by proteases in vitro. (A) is a western blot gel showing S protein cleavage by the CatL in the presence and absence of the wild type SerpinB3 (B3-WT) after 5 h incubation. (B) is a western blot gel showing S protein cleavage by the TMPRSS2 in the presence and absence of SerpinB3 variants (B3-WT and B3-TMP) after overnight incubation. (C) is a western blot gel showing S protein cleavage by the furin in the presence and absence of SerpinB3 variants (B3-WT and B3-Furin) after overnight incubation. S protein containing a C-terminal Flag tag was assessed by anti-Flag antibody. Representative data from three independent experiments are shown.

FIG. 4 are graphs showing the SerpinB3 variants inhibition of lentiviral S-pseudoparticles cell entry and SARS-CoV-2 infection and replication. (A) is a graph showing the proportion A549-ACE2-TMPRSS2 cells in which the S-pseudoparticles have penetrated (%). The cells were incubated for 1 h with media (control) or with increasing concentrations of SerpinB3 variants or camostat mesylate (CM), followed by infection with SARS-CoV-2 S pseudoparticles (in 1:1 ratio). Luciferase activities (representing pseudoparticle entry) were analysed in cell lysates at 48 h post-pseudoparticle infection. (B) is a graph showing the proportion of cells infected by SARS-CoV-2(%). VeroE6/TMPRSS2 cells were treated with increasing concentrations of SerpinB3 variants before SARS-CoV-2 infection (pre-treatment), 18 h post-infection (post-treatment), and both (full treatment). The percentage of SARS-CoV-2 infected cells was determined using flow cytometry detecting virus nucleocapsid staining. The mean±SEM from n=2 (A) and n=3 (B) independent experiments in triplicates are shown (2-way ANOVA with Tukey's comparison test).

FIG. 5 is a graph showing the cell viability assay of VeroE6/TMPRSS2 cells after incubation with SerpinB3 variants. VeroE6-TMPRSS2 expressing cells were assessed according for their viability after 18 h at the indicated concentrations of Serpin variants, A1AT or CM. DMSO (20%) was used to induce death as a positive control. Results are presented as the mean±SE of 2 independent experiments performed in 6 replicates. 2-way ANOVA with Tukey's comparison test: *** P<0.0005. A1AT, alpha-1 antitrypsin. CM, Camostat mesylate.

FIG. 6 are graphs comparing the inhibitory activities of B3-TMP to A1AT. (A) is a graph showing the activity of the proteases TMPRSS2, trypsin, chymotrypsin and elastase following incubation with either B3-TMP, A1AT or a control. The means±SE from n=3 independent experiments. Statistical analysis was undertaken using a one-way ANOVA with Tukey's comparison test. (B) and (C) are graphs showing the second order inhibition rate constant (k₂) for the inhibition of TMPRSS2 by either (B) A1AT or (C) B3-TMP. The residual initial velocity of substrate cleavage by TMPRSS2 (0.1 μM) was determined over time following incubation with various concentration of either inhibitors. The natural logarithm of the residual activity (ln(E)) was plotted against the time points and the data was fit with linear regression analysis. The slopes of these lines represent −k_{obs}. This k_{obs} is replot against serpin concentration to yield the second order inhibition rate constant (k₂).

FIG. 7 are graphs comparing the B3-TMP variant and A1AT in suppressing lentiviral S-pseudoparticle mediated cell entry and SARS-CoV-2 infection. (A) is a graph showing the proportion of A4549-ACE2-TMPRSS2 cells which the lentiviral S-pseudoparticle has entered (%) in the presence of either A1AT or B3-TMP. The A1AT and B3-TMP were added to the A549-ACE2-TMPRSS2 cells 1 h prior to infection with pseudoparticles and along with the pseudoparticles. (B) is a graph showing the percentage of SARS-CoV-2 infected cells in the presence of either A1AT or B3-TMP. VeroE6/TMPRSS2 cells were treated with either A1AT or B3-TMP 1 h before, simultaneously with, and overnight after infection with SARS-CoV-2. (C) is a graph showing the effect of the protease inhibitors on SARS-CoV-2 infection in three different treatment conditions. B3-TMP, A1AT, DMEM and CM were added as ‘Pre-treatment’ (1 h before infection and maintained for 1 h infection), ‘Post-treatment’ (added after the infection, when the virus has been removed) and ‘Full-treatment’ (both pre-treatment and then post-treatment with fresh drug) in VeroE6/TMPRSS2 cells. The mean±SEM from n=2 (A and C) and n=3 (B) independent experiments in triplicates are shown (2-way ANOVA with Tukey's comparison test).

FIG. 8 is a graph comparing B3-WT, B3-TMP and B3-Furin in suppressing infection by either wild type SARS-CoV-2 (D614G) or the Omicron (BA.5) SARS-CoV-2 variant. VeroE6/TMPRSS2 cells were treated with increasing concentrations of each of SerpinB3 variants, the small molecule furin inhibitor MI-1851 (FI) or camostat mesylate (CM) 1 h before, simultaneously with, and 18 h post-infection with either variant of SARS-CoV-2. The percentage of SARS-CoV-2 infected cells was determined using flow cytometry detecting virus nucleocapsid staining. The mean±SEM from n=2 (A) and n=3 (B) independent experiments in triplicates are shown (2-way ANOVA with Tukey's comparison test).

### DETAILED DESCRIPTION OF THE INVENTION

The following examples present a description of various specific aspects of the intended invention, and are not presented to limit the intended invention in any way.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

### EXAMPLES

### Mutagenesis of SerpinB3

The full-length open reading frame cDNAs for SerpinB3 (SCCA-1) had previously been cloned into the pRSETC (Invitrogen) expression vector. Using this plasmid containing wild-type sequence (GenBank: AB046399.1) as a template, RCL alterations were performed using Quikchange® Site Directed Mutagenesis, where primers were designed with extensions up to 20 nucleotides to replace RCL residues as shown in Table 1. PCR products were Dpn1 treated and initially transformed into DH5a competent cells. Following sequence verification (Eurofins) plasmids were transformed into E. coli BL21(DE3) cells (NEB).

TABLE 1
Primer sequences for the
generation of SerpinB3 variants.

| | | | SEQ ID |
|---|---|---|---|
| Variant | | Sequence | No. |
| B3- | Forward | 5′ atc caa ttt aga gtc | 1 |
| TMP | | tca cct gct tca act aat | |
| | | gaa 3′ | |
| | Reverse | 5′ gac tct aaa ttg gat | 2 |
| | | tac agc ggt ggc agc tgc | |
| | | agc 3′ | |
| B3- | Forward | 5′ gta cgc aat tca cgc | 3 |
| Furin | | tca tca cct gct tca act | |
| | | aat gaa 3′ | |
| | Reverse | 5′ tga gcg tga att gcg | 4 |
| | | tac tac agc ggt ggc agc | |
| | | tgc agc 3′ | |

### Expression and Purification of Recombinant Proteins

Transformed E. coli BL21(DE3) cells were grown in Overnight Express autoinducing medium (Formedium) containing 100 μg/ml ampicillin for 16 h at 37° C. Cells were harvested by centrifugation at 4000 g for 20 min and lysed using B-PER lysis reagent (ThermoScientific). Soluble material was clarified by centrifugation of the lysate at 12,500 g for 20 min at 4° C., followed by 0.22 m filtration. The filtrate was applied to Q-Sepharose (Sigma) resin equilibrated with 50 mM Tris-HCl pH 7.0, as a negative ion-exchange step to remove E. coli proteins. The unbound fraction was applied to Chelating Sepharose Fast Flow resin (GE Healthcare) pre-charged with 50 mM NiSO₄, and equilibrated with 150 mM NaCl, 50 mM Tris-HCl pH 7.9. Following 5 column volume washes with this buffer containing 20 mM imidazole, proteins were eluted with 0.5 M imidazole, and buffer exchanged into either PBS or DMEM for further analysis. Prior to cell infection experiments, endotoxins were removed using Endotoxin removal columns (Thermo Scientific) according to manufacturer instructions.

### Protease Inhibition Assays

By monitoring the linear rate of cleavage of fluorogenic substrates, protease specificity of purified proteins was assessed. CatL was gifted by Dr Matej Vizovišek, Jozef Stefan Institute, Slovenia. TMPRSS2 was obtained from Cusabio Technology and furin from BioLegend.

Proteases were incubated with a 1:5 molar excess of serpin and residual activity was determined by cleavage of fluorescent substrates. All assays were carried out at room temperature in 96-well plates in a total volume of 100 μL. Residual proteolytic activity was measured using a SpectraMax M3 Microplate Reader (Molecular Devices, Inc), using the appropriate buffer and synthetic fluorogenic peptide substrate: Cathepsin L, Z-FR-AMC (10 μM), 50 mM Na-Acetate, 100 mM NaCl, 1 mM EDTA, pH 5.5; Chymotrypsin, Z-AAPF-AMC (5 μM), PBS, 1 mg/ml BSA; Elastase, Met-O-Suc-AAPV-AMC (10 μM), 50 mM Tris, 120 mM NaCl, pH 8.0, 1 mg/ml BSA; Furin, Boc-RVRR-AMC (50 μM) 20 mM Tris, 1 mM CaCl₂), 0.5% Brij-35, pH 9; TMPRSS2, Boc-QAR-AMC (80 μM), PBS; Trypsin, Z-FR-AMC (10 μM), 50 mM Tris, 0.2 mM DTT, 1 mg/ml BSA. The excitation and emission wavelengths used were 380 nm and 460 nm respectively. All assays were performed in triplicate.

For kinetic analysis of TMPRSS2 inhibition, second order inhibition rate constants were determined under pseudo first-order conditions using a range of serpin concentrations, and with TMPRSS2 buffer and substrate detailed above. Reactions were performed at room temperature and residual activity was determined at intervals over a 10 min incubation with a molar excess of serpin over protease. Concentration ranges were 0.12 to 0.44 μM for B3-TMP and 1.0 to 4 μM for A1AT. The apparent first order rate constant k_{obs} for each concentration was calculated from the slope of the natural log of residual activity over time (−k_{obs}). These values were plotted against inhibitor concentration and linear regression analysis of this plot gave the second order inhibition rate constant k₂, with standard error determined.

### Spike Protein Cleavage

Recombinant spike protein UniProt ID P0DTC2 with both His & Flag Tag (GenScript, Z03481) consisted of the extra cellular domain (ECD) with the sequence Val16-Pro1213 fused to poly His and Flag tags at the C-terminus. S protein, in the presence and absence of purified recombinant serpin was incubated with either furin, TMPRSS2 or CatL in the appropriate assay buffer as above. After 24 h (or 5 h for CatL cleavage) products were analysed by immunoblot with monoclonal anti-FLAG antibody (1:2000; Merck). The protein band was visualized using Vilber Fusion imaging system following incubation with anti-mouse IgG, HRP-linked secondary antibody (1:5000; Cell Signaling; 70765).

### Cell Culture

A549-ACE2-TMPRSS2 cells (gifted from Prof. Suzannah J Rihn) were maintained as described. Vero-E6/TMPRSS2 cells (#100978) were obtained from the Centre For AIDS Reagents (CFAR), the National Institute for Biological Standards and Control (NIBSC). Cells were cultured in Dulbecco's modified Eagle medium (DMEM Thermo Scientific, 61965-026) supplemented with GlutaMAX™ and 10% (DMEM-10) or 2% (DMEM-2) foetal bovine serum (FBS) (Themo Scientific, 10500-064) and Geneticin (Thermo Scientific, 10131035) at a concentration of 1 mg/ml, at 37° C. in 5% CO₂. Cells routinely tested negative for mycoplasma.

### Generation of Lentiviral Pseudoparticles

Either SARS-CoV-2 Spike pseudoparticles (Spp) or no-glycoprotein (NE) pseudoparticle controls were produced in HEK293T cells. Cells were seeded in 100 mm dishes at 4×10⁶/well 24 h prior to transient transfection with three plasmids; 3.545 μg P8.91 (encoding for HIV-1 gag-pol), 3.545 μg CSFLW (lentivirus backbone expressing a firefly luciferase reporter gene), and 150 ng of either SARS-CoV-2 Spike (Accession No MN908947.3, with Spike coding sequence cloned in pcDNA3.1) or NE (empty plasmid, pcDNA3.1). Transfected cells were incubated at 37° C., 5% CO₂ and after 24 h, the transfection mix was replaced with DMEM containing 10% (v/v) FBS. Harvested supernatants containing Spp or NE pseudoparticles were taken at 48 h and 72 h post-transfection, pooled, and centrifuge at 1,200 g for 10 min at 4° C. to remove cellular debris, aliquoted and stored at −80° C.

### Assay for Pseudoparticle Cell Entry Inhibition

A549-ACE2-TMPRSS2 cells were seeded in 96-well plate at 3.75×10⁴/well one day prior to treatment and transduction. Prior to transduction with Spp or NE, cells were treated with 100 μl of serpin variants for 1 h, then media removed and replaced with 200 μl of either Spp or NE mixed 1:1 with the serpin variants and cells incubated for 48 h at 37° C., 5% CO₂. To quantify firefly luciferase, cells were lysed in 50 μl/well Passive Lysis Buffer (Promega), and cell lysates were analyzed for luciferase activity using a luminometer and the firefly luciferase activity produced by Spp was normalised to NE control. SerpinB3 variants were tested in three technical replicates and two independent experiments.

### SARS-CoV-2 Clinical Isolates

Live SARS-CoV-2 experiments were carried out in Containment Level 3 laboratory under Biosafety Level 3 guidelines. WT clinical isolate with D614G substitution (CEPHR_IE_B.177.18_1220, GenBank accession ON350866, Passage 1) and clinical isolates of the Omicron BA.5 variant, Pango lineage (GenBank accession OP508004, Passage 1) were isolated on Vero-E6/TMPRSS2 cells from SARS-CoV-2 positive nasopharyngeal swabs. Viral RNA was isolated using Qiagen Viral mRNA mini kit according to manufacturer instructions. Viral RNA genome was sequenced to confirm the integrity of the Spike protein. The titres of the virus stocks were determined by plaque assay. WT (D641G) had a titre of 2.6×10⁶ Plaque Forming Units (PFU) per ml.

### VeroE6-TMPRSS2 Viral Infection Assay

Vero-E6/TMPRSS2 cells were plated (2.5×10⁴ cells per well) in 96-well clear flat-bottom plates and incubated overnight at 37° C. 5% CO₂ to reach 90-100% confluency at time of infection. Drug Dose Response of SerpinB3 variants, α-1 antitrypsin (A1AT), the small molecule furin inhibitor, MI-1851 (FI) and camostat mesylate (CM) were performed using 4 points, and 2-fold dilutions from 50 μM stock, as indicated. Following 1 h pre-treatment with drugs in DMEM-2 at 37° C. 5% CO₂, WT (D614G) SARS-CoV-2 (520 PFU per well of 2.5×10⁴ cells for a Multiplicity of Infection (MOI) of 0.02) and Omicron BA.5 SARS-CoV-2 (520 PFU per well of 2.5×10⁴ cells for a Multiplicity of Infection (MOI) of 0.02) was added to the cells in drug-containing DMEM-2 for 1 h at 37° C. 5% CO₂. Cells were then washed twice in dPBS (Thermo Scientific, 14190094) and further incubated in DMEM-2 containing drugs for 18 h at 37° C. 5% CO₂. Drugs were tested in technical triplicate with a minimum of three independent experiments.

For time-of-addition experiments, cells were exposed to protease inhibitors (50 μM) or control in three drug addiction schemes run concurrently. “Pre-treatment”: drug treatment was limited to 1 h pre-treatment and 1 h during viral exposure. “Post-treatment” treatment: following dPBS washes drugs were added immediately post-infection and for 18 h. “Full-treatment” treatment included both pre-treatment and then post-treatment as describe above. Drugs were tested in three technical replicates and two independent experiments.

### Flow Cytometry Analysis of SARS-CoV-2 Infected Cells

At 18 h post-infection, cells were washed in dPBS, and trypsinised (Trypsin-EDTA, Thermo Scientific, 15400054) to achieve single cell suspension. Cells were fixed in 4% formaldehyde solution (Sigma Aldrich, F8775) in the dark at room temperature for a minimum of 8 h. All steps-post fixation were carried out in Biosafety Level 2 conditions in Class 2 Biosafety Cabinets. Cells were permeabilised with Perm/Wash Buffer (BD, 554723) according to manufacturer's instructions, which was maintained throughout antibody staining. Intracellular SARS-CoV-2 Nucleoprotein (NP) staining was performed with SARS/SARS-CoV-2 Nucleocapsid Monoclonal Antibody (E16C) (1/100 dilution, Invitrogen, MA1-7403) and goat anti-mouse IgG2b-FITC (1/500 dilution, Santa Cruz Biotechnology, SC-2080). Cells were resuspended in 60 μl PBS-EDTA-2% (Sigma-Aldrich, 03690-100 ml) for flow cytometry analysis (CytoFlex S, Beckman Coulter). Cells were gated with Forward and Side-Scatter gates to exclude debris from intact cells, then single cells were gated based on linearity between Area and Height. % infected cells analysed using the FITC-channel (Blue 488 nM laser, 525/40 laser). Analysis was performed using CytExpert software (version 2.4.0.28, Beckman Coulter). The mean % infected cells were determined from the technical replicates and normalised to the positive (virus alone) and negative (DMEM-2) controls to determine the % of viral inhibition.

### Cell Viability Assay

Vero-E6/TMPRSS2 cells were plated (2.5×10⁴ cells per well) in 96-well clear flat-bottom plates and incubated overnight at 37° C. 5% CO₂ to reach 90-100% confluency at time of infection. Dilution of serpin variants, A1AT, FI, and CM were performed using 4 points, and 2-fold dilutions from 50 μM stock, as indicated. Cells were pre-treated with 100 μl drugs for 2 h at 37° C. 5% CO₂. 100 μl DMEM-2 was then added to the wells and cells were incubated overnight at 37° C. 5% CO₂. Cell death was forced in triplicate wells using by incubating cells in 20% DMSO solution for 10 min at 37° C. 5% CO₂. Supernatant was removed, cells washed twice in dPBS before staining with Viobility™ 405/452 Fixable Dye (130-130, 1/100 dilution, Miltenyi Biotech), according to manufacturer's instructions. Cells were washed twice in dPBS, trypsinised and fixed in 4% formaldehyde solution. Cells were resuspended in 60 μl PBS-EDTA-2% for flow cytometry analysis (CytoFlex S, Beckman Coulter). Cells were gated with Forward and Side-Scatter gates to exclude debris from intact cells, then single cells were gated based on linearity between Area and Height. Viobility stained cells were detected in the PB450 channel (Violet 405 nM laser, 450/45 filter). Negative control (DMEM alone) was used to set the boundary of the live cells. Positive control (DMSO treated) was used to ensure uptake of the stain by dead cells. Analyses were performed using CytExpert software (version 2.4.0.28, Beckman Coulter). The mean % dead cells were determined from the technical replicates and presented as the % viable cells per condition (100%-% dead cells).

### Statistical Analysis

All the statistical analysis was performed using Minitab version 18. For statistical analysis, either ordinary one-way ANOVA or 2-way ANOVA with Tukey's comparison test was used as indicated in figure references above.

### Novel SerpinB3 RCL Mutagenesis

Novel inhibitors to block SARS-CoV-2 cell entry were designed using a recombinant serpin, SerpinB3 as a template. SerpinB3, or SCCA-1, is a 44.5 kDa cross-class inhibitor of cysteine proteases, including the target cathepsin L, CatL. The closely related SerpinB4 (SCCA-2) inhibits the serine proteases chymotrypsin and cathepsin G, and it has been shown that RCL loop swaps could change the specificity of B3 or B4 effectively [1]. Therefore, the SerpinB3 backbone is not protease-class specific, and can be an effective template scaffold to target proteases with either serine or cysteine active site nucleophiles.

A construct was designed to inhibit TMPRSS2 (B3-TMP). The RCL mutations selected were based on substrate profiling of preferred residues using a combinatorial peptide library [2] with preferred amino acids (IQFRV) substituted for residues 350 to 354 in SerpinB3 i.e., positions P4 to P1′ (IQFRV). A subsequent study combining rational ketobenzothiazole inhibitor design and multiplex substrate profiling by mass spectrometry largely, confirmed these subsite preferences, with a strong preference for phenylalanine indicated in the P2 position [3].

A second construct was designed comprising a chimera with the SerpinB8 sequence of P4 to P1 replacing the SerpinB3 sequence (B3-Furin). SerpinB8 (human proteinase inhibitor 8, PI8) is an effective inhibitor of furin [4] and previous loop swap studies with antitrypsin defined a P6 to P1 insertion (VVRNSR) generating a chimeric antitrypsin Portland (al-PDX) with a k₂ for furin comparable to native SerpinB8 [5]. The SerpinB3 RCL sequence already contains the two valine residues, so the three P1 to P3 residues were replaced with the 4 residues RNSR. This insertion also increased the RCL length to the majority superfamily consensus of 14 residues (from P1 to the hinge region residue P14 alanine) where native SerpinB3 has just 13 residues.

### Inhibition of Cathepsin L and TMPRSS2 and Furin by SerpinB3 Variant

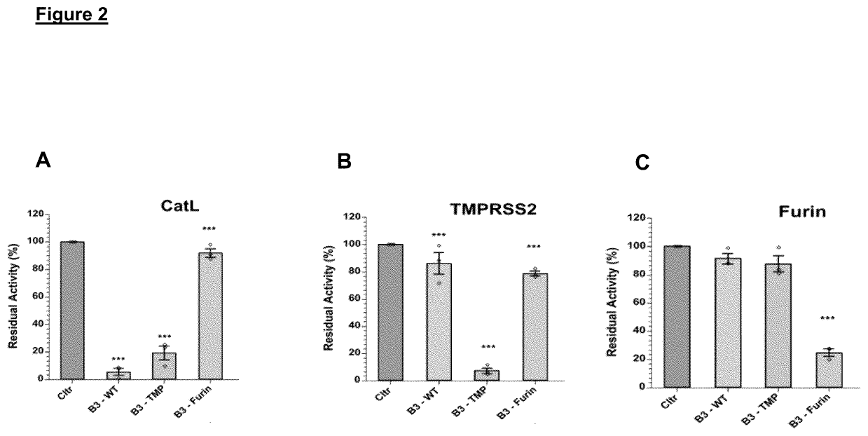

Proteins were expressed and purified by ion exchange and IMAC chromatography. Inhibitory activity was assessed by examining fluorometric substrate cleavage following incubation of the protease with an excess of serpin. The RCL mutations made caused a loss of CatL inhibitory activity, although the B3-TMP variant retained significant CatL inhibition at 81% compared to 95% with native SerpinB3 under identical selected conditions as shown in FIG. 2A. For TMPRSS2 inhibition at a 5:1 serpin:protease ratio, the only effective variant was the B3-TMP construct where 92% inhibition of TMPRSS2 was achieved as illustrated in FIG. 2B. The B3-Furin variant was the most effective at inhibiting furin as shown in FIG. 2C.

### SerpinB3 Variants can Prevent SARS-CoV-2 Spike Degradation In Vitro

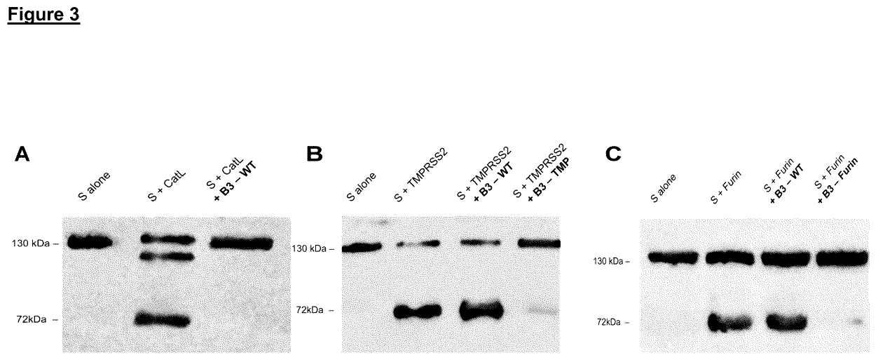

The ability of the SerpinB3 variants to inhibit cleavage of the viral substrate in vitro were examined using the recombinant SARS-CoV-2 spike protein extracellular domain (1273aa) tagged with Flag and 6×His (S protein sequence of original Wuhan variant, P0DTC2). Overnight incubation of S protein with both TMPRSS2 and furin or 5 h incubation with CatL resulted in cleavage with a C-terminal Flag tagged product detected by immunoblot as illustrated in FIG. 3.

B3-TMP was able to block S cleavage by the corresponding host protease, with little or no cleavage products visible and the 135 kDa spike protein substrate remaining intact. The CatL products of approximately 105 kDa and 72 kDa suggest an additional cleavage site in the protein compared with furin and TMPRSS2. This is consistent with the recent findings of novel CatL cleavage sites at position 259 (CS-1) and at position 636 (CS-2) [9].

### SerpinB3 and Modified Variants Inhibit SARS-CoV-2 Spike Mediated Pseudovirus Cell Entry

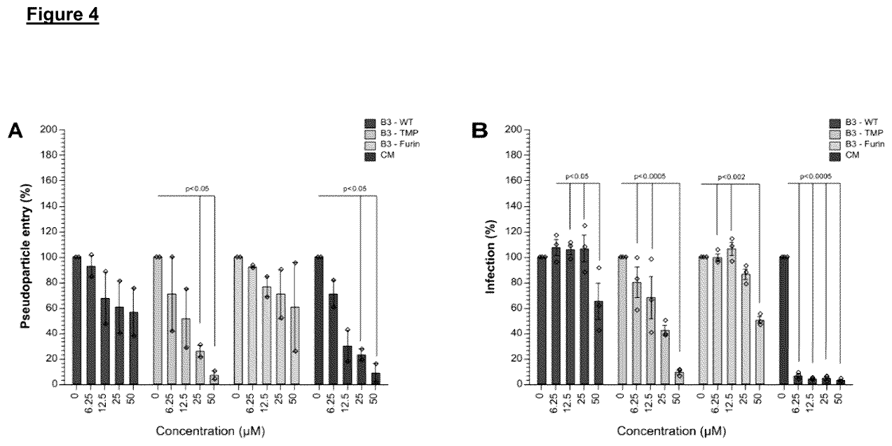

The ability of the WT, B3-TMP and B3-furin to prevent SARS-CoV-2 S-pseudoviral particle cell entry was investigated. Pseudoviral particles expressing S protein were added to A549 cells expressing ACE2 and TMPRSS2. Camostat mesylate (CM), an inhibitor of TMPRSS2, was included as a positive control. Concentrations ranging from 6.25 μM to 50 μM were tested, and luciferase activity was measured to analyze pseudoparticle entry. All 3 serpin constructs exhibited concentration-dependent inhibition of pseudoparticle entry, with the anti-TMPRSS2 variant (B3-TMP) having the greatest effect, showing 90% inhibition at 50 μM, which is comparable to the CM control as shown in FIG. 4A.

### SerpinB3 Anti-TMPRSS2 Inhibits SARS-CoV-2 Infection in VeroE6-TMPRSS2 Cells

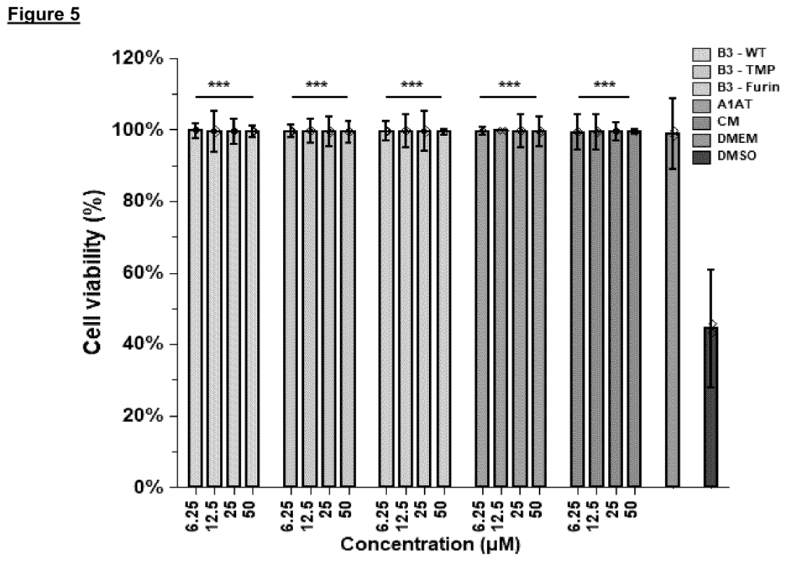

To examine the capacity of the SerpinB3 to inhibit SARS-CoV-2 replication, an infection model using Vero-E6/TMPRSS2 cells and SARS-CoV-2 (WT, D614G) was employed where the percentage of SARS-CoV-2 infected cells was determined at 18 h post-infection (pi) by flow cytometry analysis of nucleocapsid protein positive cells as illustrated in FIG. 4B. Cell viability assays showed that all SerpinB3 variants and CM were not cytotoxic as shown in FIG. 5.

All constructs showed some inhibitory activity over control. For B3-WT (inhibiting CatL) and B3-Furin (inhibiting furin), only the higher concentration (50 μM) limited SARS-CoV-2 replication by 50%. However, the construct designed to inhibit TMPRSS2 (B3-TMP) displayed a good dose-dependent downregulation of viral replication, achieving up to 50% inhibition at 6.5 M and up to 92% inhibition at 50 μM. These results are consistent with previous findings that SARS-CoV-2 entry is preferentially facilitated via membrane fusion at the cell surface following TMPRSS2-mediated Spike processing in this cellular model and less via the endosomal route and cathepsin L processing [6]. A further interpretation is that the B3-TMP serpin variant is inhibiting both pathways, considering the retention of cathepsin L inhibition in addition to the gain in TMPRSS2 inhibition as illustrated in FIG. 2.

SerpinB3 Anti-TMPRSS2 (B3-TMP) is More Effective than A1AT at Inhibiting TMPRSS2 and Suppressing SARS-CoV-2 Infection

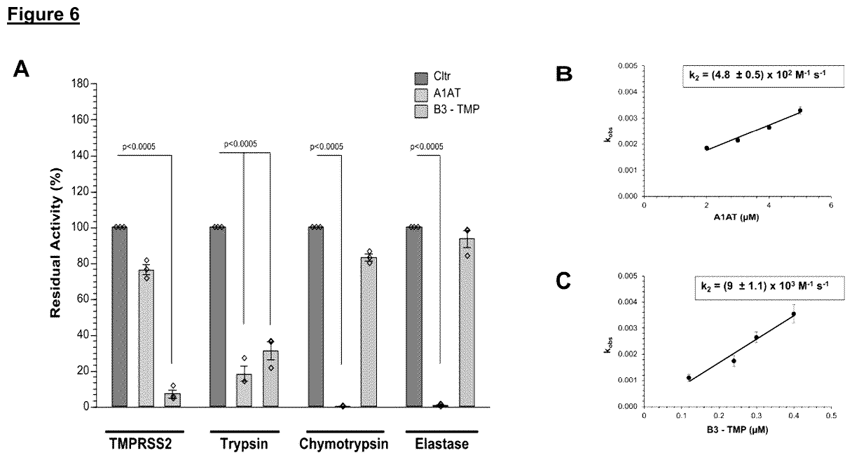

The serpin A1AT has been shown to inhibit TMPRSS2 activity and SARS-CoV-2 infection, and therefore its inhibitory activity was compared with B3-TMP. As an indication of target protease selectivity, both serpins were tested with mammalian trypsin, chymotrypsin and elastase, in addition to TMPRSS2. A short preincubation time of 10 minutes prior to addition of fluorogenic substrate was used and residual activity determined. A1AT was the more effective inhibitor of trypsin, chymotrypsin and pancreatic elastase, but for TMPRSS2 activity B3-TMP gave 92% inhibition where A1AT exhibited 15% inhibition as shown in FIG. 6A.

Wettstein et al [7] reported effective inhibition of recombinant TMPRSS2 by Prolastin, a pharmaceutical preparation of A1AT from human plasma, (analysed as <62% purity [8]). In that study residual rTMPRSS2 activity was measured over an extended 3 h incubation with serpin and substrate, in contrast to the present study, which observed rapid loss of activity following incubation with serpin with real-time hydrolysis monitored. In the current study a kinetic comparison was also performed, where second order rate constants of TMPRSS2 inhibition were determined under pseudo first-order conditions using a range of serpin concentrations. The respective k₂ values of (4.8±0.55)×10² M⁻¹ s⁻¹ for A1AT as shown in FIG. 6B and of (9±1.1)×10³ M⁻¹ s⁻¹ for B3-TMP as shown in FIG. 6C indicate an 18-fold increase in TMPRSS2 specificity for B3-TMP over A1AT.

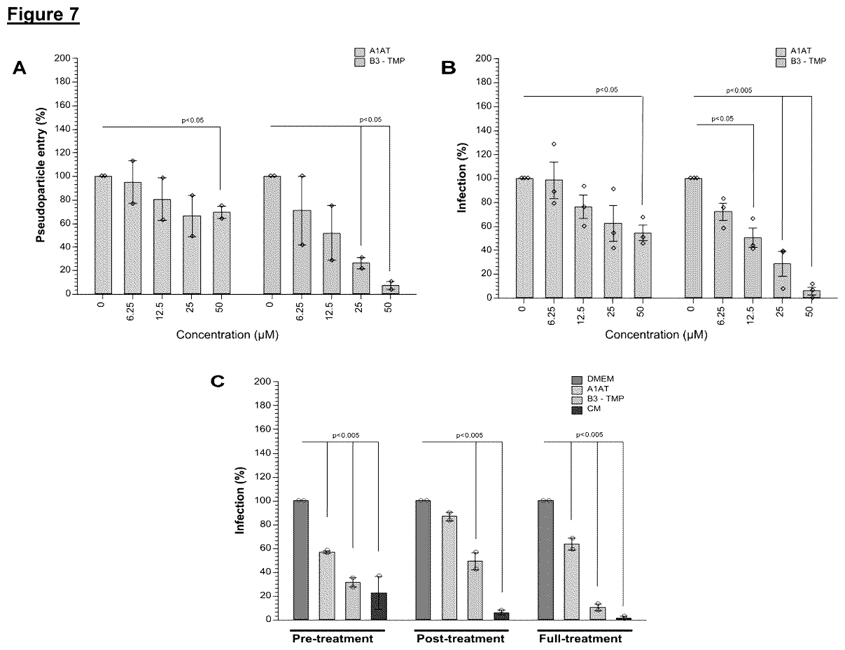

Inhibition of SARS-CoV-2 spike mediated pseudoparticle entry was also examined, and the B3-TMP variant was found to be more effective at suppressing entry of pseudoparticles in A549-ACE2-TMPRSS2 cells than A1AT as shown in FIG. 7A. Here, A1AT reach plateau at 12.5 μM with 40% inhibition while B3-TMP achieved 70% inhibition at 12.5 μM and 90% inhibition at 50 μM. Similarly, for SARS-CoV-2 entry into VeroE6-TMPRSS2 cells, B3-TMP was more effective at inhibiting viral entry at equivalent concentrations to A1AT as shown in FIG. 7B, with 92% inhibition at 50 μM compared to 45% for 50 μM A1AT.

To delineate how the protease inhibitors exerted their antiviral effects, a ‘Time-of-Addition’ experiment was undertaken with 50 μM B3-TMP and A1AT proteins. These were added either before and at the time of viral entry (1 h prior and during 1 h of viral infection), post-treatment (18 h post-infection) or for full-time treatment where inhibitors are added before, during infection, and post-infection for 18 h following virus removal. As seen in FIG. 7C, full time treatment of B3-TMP is the more potent with 93% inhibition of viral replication (as described above). Interestingly, B3-TMP treatment limited to viral entry remains effective to inhibit SARS-CoV-2 and achieved up to 70% of viral inhibition, strongly supporting that B3-TMP interferes with viral entry at all times of addition with best results for continuous presence of serpin inhibitor. As above, A1AT displayed a similar pattern of viral inhibition albeit less potent supporting that A1AT inhibits viral entry as well which is consistent with the findings of Wettstein et al [11]. The post-entry effect suggested that the B3-TMP serpin can block new rounds of infection of progeny virus into uninfected cells.

SerpinB3 Anti-TMPRSS2 (B3-TMP) is More Effective than SerpinB3 WT at Suppressing SARS-CoV-2 WT (D614G) and SARS-CoV-2 Omicron BA.5 SARS-CoV-2

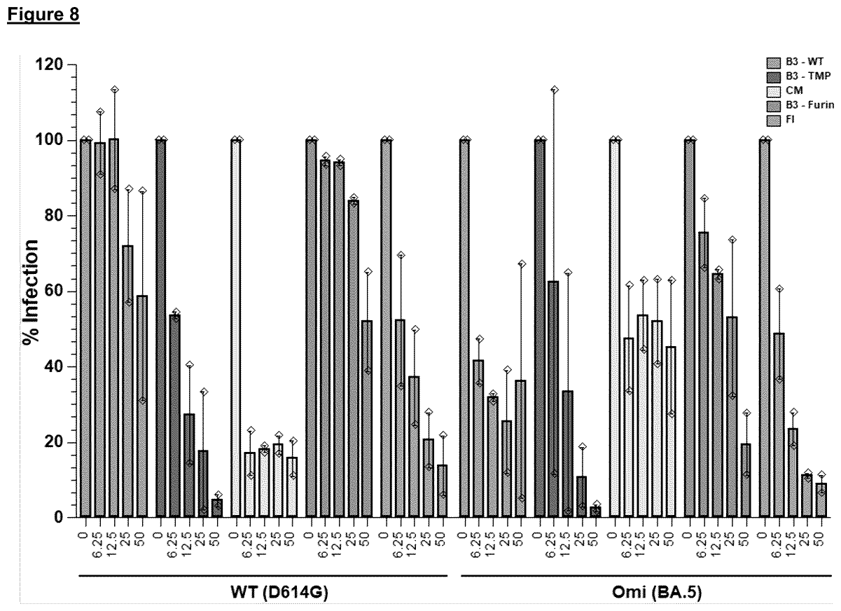

To examine the capacity of the SerpinB3 to inhibit different variants of SARS-CoV-2 infection, an infection model using Vero-E6/TMPRSS2 cells and two variants of SARS-CoV-2 (WT (D614G) and Omicron BA.5) was employed where the percentage of SARS-CoV-2 infected cells was determined at 18 h post-infection (pi) by flow cytometry analysis of nucleocapsid protein positive cells (FIG. 8). CM and a small molecule furin inhibitor (MI-1851) were used as controls.

Both WT-SerpinB3 and B3-TMP showed reductions in SARS-CoV-2 infection of both variants compared to the controls and the reduction was greater with the Omicron BA.5 variant compared to the WT (D614G) variant. B3-TMP was a greater inhibitor for both viral strains than WT-SerpinB3.

### DISCUSSION

The aim of these experiments was to establish a common serpin scaffold that could be modified to specifically inhibit TMPRSS2. The RCL residues in SerpinB3 were mutated using data from the substrate peptide library of Lucas et al [2], which indicated the preferred TMPSS2 amino acids (IQFRV). These residues replaced RCL residues 350 to 354 (positions P4 to P1′). The success of this as seen in FIG. 2B was not a certainty and previous attempts using this approach for A1AT report poor translation from such libraries [9]; there was a possibility that the modified serpin would result in the kinetic pathway favoring the substrate route rather than a complex formation, inhibitor pathway, and this has been explored using a Conserpin, a synthetic consensus serpin scaffold [10]. The inhibitors of the present invention are believed to be the first successful example of a modified serpin solely based on substrate peptide library data.

During the experiments, a 92% loss of TMPRSS2 activity was observed following a 10-minute incubation with the modified SerpinB3-TMP, a time frame consistent with the rapid suicide mechanism of serpins. The B3-TMP showed better selectivity for TMPRSS2 than A1AT as illustrated in FIG. 6A, and kinetic comparisons demonstrate that B3-TMP has a second order rate constant for TMPRSS2 inhibition that is 18-fold greater than A1AT as shown in FIGS. 6B and 6C.

B3-TMP was shown to reduce S protein cleavage in vitro as shown in FIG. 3 and reduce SARS-CoV-2 (D614G) pseudoparticle entry and infection. Recent studies on the Omicron variants of SARS-CoV-2 report an entry route less dependent on TMPRSS2 spike cleavage and suggest that the CatL pathway may be more important. Therefore, it was expected that the B3-WT, which targets CatL, would be a more effective inhibitor of the Omicron BA.5 variant. Surprisingly and advantageously, however, the B3-TMP variant was shown to inhibit Omicron BA.5 infection to a greater extent than the B3-WT. Given the B3-TMP variant retains significant inhibition of CatL as shown in FIG. 2A, it is believed that a potential dual-target effect may suppress cell entry.

As naturally occurring inhibitors present in plasma and other tissues at concentrations up to 40 μM, serpins have low immunogenicity and toxicity, and with minor RCL residue changes as illustrated in this study, modified serpins are strong candidates for evaluation in viral infection. Unlike therapeutics that block the specific viral encoded main M-Pro protease, inhibitors of host cell surface proteases may have a broader antiviral potential for both influenza and coronavirus infections.

Although the amino acid sequence for TMPRSS2 specificity used in this study was IQFRV, the inventors believe that alternative RCL variations would produce similar effects. However, glutamine at P3 and arginine at P1 are estimated to be the most critical residues for TMPRSS2 specificity, followed by phenylalanine at P2 and any hydrophobic residues at P4 and P1′.

In addition to viral infection, TMPRSS2 is also a potential target for cancer therapy with well-documented links to prostate cancer metastasis, where a TMPRSS2-ERG fusion gene is found in 50% of prostate tumours [12]. Therefore, TMPRSS2 is an attractive target for the treatment of both viral infections and tumours.

The foregoing embodiments are not intended to limit the scope of protection of the claims, but rather to describe examples of how the invention may be put into practice.

### BIBLIOGRAPHY

The following references are incorporated herein in their entirety.
[1] Schick C, Bromme D, Bartuski A J, Uemura Y, Schechter N M, Silverman G A. The reactive site loop of the serpin SCCA1 is essential for cysteine proteinase inhibition. Proc Natl Acad Sci 1997; 95: 13465-13470.
[2] Lucas J M, Heinlein C, Kim T, Hernandez S A, Malik M S, True L D, Morrissey C, Crey E, Montgomery B, Mostaghel E et al. The androgen-regulated protease TMPRSS2 activates a proteolytic cascade involving composnents of the tumour microenvironment and promotes prostate cancer metastasis. Cancer Discov. 2014; 4: 1310-1325.
[3] Mahoney M, Damalank V C, Tartell M A, Chung D H, Lourengo A L, Pwee D, Bridwell A E M, Hoffmann M, Voss J, Karmarkar P et al. A novel class of TMPRSS2 inhibitors potently blocks SARS-CoV-2 and MERS-CoV viral entry and protect human epithelial lungs. Proc. Nat. Acad. Sci. 2021; 118: e2108728118
[4] Dahlen J R, Jean F, Thomas G, Foster D C, Kisiel W. Inhibition of soluble recombinant furin by human proteinase inhibitor 8. J. Biol. Chem. 1998; 273: 1851-1854.
[5] Izaguirre G, Qi L, Lima M, Olson S T. Identification of serpin determinants of specificity and selectivity of furin inhibition through studies of alpha1PDX (alpha1-protease inhibitor Portland)-serpin B8 and furin active-site loop chimeras. J Biol. Chem. 2013; 288: 21802-21814.
[6] Koch J, Uckeley Z M, Doldan P, Stanifer M, Boulant S, Lozach P. TMPRSS2 expression dictates the entry route used by SARS-CoV-2 to infect host cells. EMBO J. 2021; 40: e107821.
[7] Wettstein L, Weil T, Conzelmann C, Müller J A, Gross R, Hirschenberger M, Seidel A, Klute S, Zech F, Bozzo C P et al. Alpha-1 antitrypsin inhibits TMPRSS2 protease activity and SARS-CoV-2 infection. Nat. Commun. 2021; 12: 1726.
[8] Cowden D I, Fisher G E, Weeks R L. A pilot study comparing the purity, functionality and isoform composition of the alpha-1 proteinase inhibitor (human) products. Curr. Med. Res. Opin. 2005; 21: 877-883.
[9] Sanrattana W, Maas C, de Maat S. SERPINs—From Trap to Treatment. Front. Med. 2019; 6: 25.
[10] Marijanovic E M, Fodor J, Riley B T, Porebski B T, Costa M G S, Kass I, Hoke D E, McGowan S, Buckle A M. Reactive centre loop dynamics and serpin specificity. Sci. Rep. 2019; 9:3870.
[11] Tanabe L M, List K. The role of type II transmembrane serine protease-mediated signalling in cancer. FEBS J. 2017; 284: 1421-1436.
[12] Mollico V, Rizzo A, Massari F. The pivotal role of TMPRSS2 in coronavirus disease 2019 and prostate cancer. Future Oncol. 2020; 16: 2029-2033.
[13] Schick C, Pemberton P A, Shi G-P, Y, Qataltepe S, Bartuski A J, Gornstein E R, Brömme D, Chapman H A, Silverman G A, Cross-Class Inhibition of the Cysteine Proteinases Cathepsins K, L, and S by the Serpin Squamous Cell Carcinoma Antigen 1: A Kinetic Analysis Biochemistry 1998; 37: 5258-5266.

Sequence Listings
SEQ ID No. 1-Base sequence for forward PCR
primer (B3-TMP):
atc caa ttt aga gtc tca cct gct tca act aat gaa
SEQ ID No. 2-Base sequence for reverse PCR
primer (B3-TMP):
gac tct aaa ttg gat tac agc ggt ggc agc tgc agc
SEQ ID No. 3-Base sequence for forward PCR
primer (B3-Furin):
gta cgc aat tca cgc tca tca cct gct tca act aat
gaa
SEQ ID No. 4-Base sequence for reverse PCR
primer (B3-Furin):
tga gcg tga att gcg tac tac agc ggt ggc agc tgc
agc
SEQ ID No. 5-Amino acid sequence of the Wild
Type SerpinB3:
MNSLSEANTKFMFDLFQQFRKSKENNIFYSPISITSALGMVLLGAKD
NTAQQIKKVLHFDQVTENTTGKAATYHVDRSGNVHHQFQKLLTEFNK
STDAYELKIANKLFGEKTYLFLQEYLDAIKKFYQTSVESVDFANAPE
ESRKKINSWVESQTNEKIKNLIPEGNIGSNTTLVLVNAIYFKGQWEK
KFNKEDTKEEKFWPNKNTYKSIQMMRQYTSFHFASLEDVQAKVLEIP
YKGKDLSMIVLLPNEIDGLQKLEEKLTAEKLMEWTSLQNMRETRVDL
HLPRFKVEESYDLKDTLRTMGMVDIFNGDADLSGMTGSRGLVLSGVL
HKAFVEVTEEGAEAAAATAVVGFGSSPTSTNEEFHCNHPFLFFIRQN
KTNSILFYGRFSSP
SEQ ID No. 6-Amino acid sequence of the
SerpinB3-TMP variant:
MNSLSEANTKFMFDLFQQFRKSKENNIFYSPISITSALGMVLLGAKD
NTAQQIKKVLHFDQVTENTTGKAATYHVDRSGNVHHQFQKLLTEFNK
STDAYELKIANKLFGEKTYLFLQEYLDAIKKFYQTSVESVDFANAPE
ESRKKINSWVESQTNEKIKNLIPEGNIGSNTTLVLVNAIYFKGQWEK
KFNKEDTKEEKFWPNKNTYKSIQMMRQYTSFHFASLEDVQAKVLEIP
YKGKDLSMIVLLPNEIDGLQKLEEKLTAEKLMEWTSLQNMRETRVDL
HLPRFKVEESYDLKDTLRTMGMVDIFNGDADLSGMTGSRGLVLSGVL
HKAFVEVTEEGAEAAAATAVIQFRVSPTSTNEEFHCNHPFLFFIRQN
KTNSILFYGRFSSP
SEQ ID No. 7-Amino acid sequence of the Wild
Type SerpinB3 RCL:
EEGAEAAAATAVVGFGSSPTST
SEQ ID No. 8-Amino acid sequence of the
SerpinB3-TMP RCL:
EEGAEAAAATAVIQFRVSPTST

What is claimed is:

1. A serine protease inhibitor (Serpin) comprising a modified Reactive Centre Loop (RCL), wherein the modified RCL comprises a transmembrane serine protease 2 (TMPRSS2) inhibitory sequence having one or more amino acid substitutions at positions P4 to P1′ of SerpinB3.

2. The inhibitor of claim 1, wherein the TMPRSS2 inhibitory sequence has up to four amino acid substitutions at positions P4 to P1′ of SerpinB3.

3. The inhibitor of claim 1, wherein the TMPRSS2 inhibitory sequence comprises an amino acid substitution at P3.

4. The inhibitor of claim 3, wherein the TMPRSS2 inhibitory sequence amino acid substitution at P3 comprises glutamine.

5. The inhibitor of claim 1, wherein the TMPRSS2 inhibitory sequence comprises an amino acid substitution at P1.

6. The inhibitor of claim 5, wherein the TMPRSS2 inhibitory sequence amino acid substitution at P1 comprises arginine.

7. The inhibitor of claim 1, wherein the TMPRSS2 inhibitory sequence comprises an amino acid substitution at P2.

8. The inhibitor of claim 7, wherein the TMPRSS2 inhibitory sequence amino acid substitution at P2 comprises phenylalanine or alanine.

9. The inhibitor of claim 7, wherein the TMPRSS2 inhibitory sequence amino acid substitution at P2 comprises phenylalanine.

10. The inhibitor of claim 1, wherein the TMPRSS2 inhibitory sequence comprises an amino acid substitution at P4 and P1′.

11. The inhibitor of claim 12, wherein the TMPRSS2 inhibitory sequence comprises a hydrophobic residue at P4 and P1′.

12. The inhibitor of claim 1, wherein the RCL sequence comprises SEQ ID No: 8 or at least about 82% identity thereof.

13. The inhibitor of claim 1, wherein the serine protease inhibitor comprises SEQ ID No: 6 or at least over about 98% identity thereof.

14. An isolated nucleic acid comprising the nucleic acid sequence encoding the serine protease inhibitor of claim 1.

15. Use of a composition comprising the polypeptide protease inhibitor of claim 1 to inhibit and/or reduce TMPRSS2 activity in a cell.

16. Use of a composition of claim 17, wherein the cell is mammalian.

17. A pharmaceutical composition comprising the serine protease inhibitor of claim 1.

18. The pharmaceutical composition of claim 19, wherein the composition is formulated for intravenous or intranasal administration.

19. A method of inhibiting TMPRSS2 activity in a cell, the method comprising administering the serine protease inhibitor of claim 1 to the cell.

20. The method of claim 19, wherein the cell is a mammalian cell.

21. A method of treating and/or preventing a condition where transmembrane serine protease 2 (TMPRSS2) activity is implicated in a subject in need thereof, the method comprising administering to the subject an effective amount of a serine protease inhibitor (Serpin) comprising a modified reactive centre loop (RCL), wherein the modified RCL comprises a TMPRSS2 inhibitory sequence having one or more amino acid substitutions at positions P4 to P1′.

22. The method of claim 21, wherein the TMPRSS2 inhibitory sequence comprises an amino acid substitution at P3 comprising glutamine.

23. The method of claim 21, wherein the TMPRSS2 inhibitory sequence comprises an amino acid substitution at P1 comprising arginine.

24. The method of claim 21, wherein the TMPRSS2 inhibitory sequence comprises a hydrophobic residue at P4 and P1′.

25. The method of claim 21, wherein the RCL sequence comprises SEQ ID No: 8 or at least about 82% identity thereof.

26. The method of claim 21, wherein the serine protease inhibitor comprises SEQ ID No: 6 or at least over about 98% identity thereof.

27. The method of claim 21, wherein the subject is a mammal, preferably a human.

28. The method of claim 21, wherein the condition is a viral infection.

29. The method of claim 28, wherein the viral infection is SARS-CoV-2.

30. The method of claim 21, wherein the condition is cancer.

31. The method of claim 30, wherein the cancer is prostate cancer.

32. The method of claim 21, wherein the serine protease inhibitor is administered to the subject intravenously or via inhalation.
